SEQUENCE LISTING (4 sequences)

SEQ ID NO: 1 - DNA, 21 bp, Artificial Sequence
  Description of Artificial Sequence Synthetic primer
gcctctcgtggcagggcagtc

SEQ ID NO: 2 - DNA, 21 bp, Artificial Sequence
  Description of Artificial Sequence Synthetic primer
catcacctggcctgaggaatc

SEQ ID NO: 3 - DNA, 23 bp, Artificial Sequence
  Description of Artificial Sequence Synthetic primer
cactgcatcaggaacaaaagcgt

SEQ ID NO: 4 - DNA, 21 bp, Artificial Sequence
  Description of Artificial Sequence Synthetic primer
catcacctggcctgaggaatc

(19) United States
(12) Patent Application Publication
(10) Pub. No.: US 2004/0241707 A1
(43) Pub. Date: Dec. 2, 2004

(54) Enhanced diagnostic potential of prostate-specific antigen expressing cells

(76) Inventors: Chun L. Gao, Gaithesburg, MD (US); Judd W. Moul, Bethesda, MD (US); Shiv Srivastava, Potomac, MD (US)

Correspondence Address: MORRISON & FOERSTER LLP, 1650 TYSONS BOULEVARD, SUITE 300, MCLEAN, VA, 22102, US

(21) Appl. No.: 10/677,495

(22) Filed: Oct. 3, 2003

(30) Foreign Application Priority Data
Apr. 1, 2002 (WO) PCT/US02/09736

(51) Int. Cl. C12Q 1/68; G01N 33/574

(52) U.S. Cl. 435/6; 435/7.23

(57) ABSTRACT

The present invention is directed to sensitive and specific methods and kits for the detection of prostate cancer in a patient. The invention uses RT-PCR to detect the expression or change in expression of PSA in epithelial cells enriched from whole blood. The methods and kits on the invention represent a a significant improvement over previous methods of CaP diagnosis. According to one embodiment, prostate epithelial cells are isolated from blood and the RNA subjected to RT-PCR. The resulting cDNA is subjected to traditional PCR amplification with primers able to distinguish between the genomic copy of the gene and the cDNA copy resulting from CaP gene expression. This method provides an assay which is more sensitive, specific and reproducible as compared to conventional methods. Results disclosed herein demonstrate a correlation between the presence of PSA-expressing prostate epithelial cells with cancer recurrence which provides a diagnostic tool for the early detection of prostate disease.

### REFERENCE TO RELATED APPLICATIONS

This application claims priority to U.S. provisional application entitled “Early Detection of PSA Expression,” serial No. 60/281,378, filed Apr. 5, 2001, which is incorporated herein by reference in its entirety.

### RIGHTS IN THE INVENTION

This invention was made, in part, with support from the United States government and the United States may have certain rights in the invention.

### BACKGROUND OF THE INVENTION

This invention relates to sensitive and specific methods for the detection of prostatic disease such as clinically organ confined prostate cancer and potential micro-metastasis. In particular, the methods relate to the use of RT-PCR (reverse-transcriptase polymerase chain reaction) to detect prostate specific antigen expression in peripheral blood derived epithelial cells. The invention also relates to kits and compositions that utilize these methods.

### DESCRIPTION OF THE BACKGROUND

Adenocarcinoma of the prostate is the most common solid tumor in American males and is the second leading cause of cancer death in men. It is estimated that 317,100 new cases of prostate cancer (CaP) will be diagnosed this year and that 41,400 deaths will be attributed to prostate cancer. Elevated serum levels of the prostate gland-specific protein, PSA, are currently the most powerful and widely used prostate cancer screening test. However, the serum PSA test is not perfect and limited due to high false positive rates. New prostate cancer specific molecular and cellular markers are clearly needed both for early and accurate detection and prognosis of prostate cancer. While several risk factors (such as age, high fat diet and race) have been identified, the cause of the increased risk has not been identified. The identification of significant risk factors has led to global recommendations that all men over 50 be screened, and all men having a familial incidence of CaP be screened beginning at age 40. These diagnostic measures have led to greater investigation of practices for screening and staging CaP with greater detection capabilities.

Demands for more accurate diagnoses and more powerful staging tools stem from the fact that prostate cancer is generally slow growing and, due to its late onset, not the primary cause of death in CaP patients. However, as statistics indicate, death will result from metastatic spread of CaP in a significant percentage of prostate cancer patients. The serum PSA screening test has revolutionized the early detection of prostate cancer. With the enormous increase in the rate of incidence of CaP, review of current practices for screening, diagnosis, and staging have been highly investigated. The majority of new cases are clinically localized without obvious metastases. However, 30-40% patients show biochemical recurrence after treatment of localized disease. More accurate detection and staging tools are needed since many prostate cancers are not cured by local therapies due to occult micrometastases. This substantial mortality from CaP could be prevented if the disease was detected before metastasis occurs. Thus, the ability to detect CaP in a subclinical stage before metastasis may be key in preventing CaP mortality. Therefore, the value of detecting circulating prostate cancer cells and their use as a staging tool is a function of the sensitivity of the test used to detect those cells.

Current methods to test for the presence of CaP begin with the analysis for prostate specific antigen (PSA) protein, which is secreted in the blood. This test focuses on the concentration of free or bound PSA in the blood. New serum PSA tests that measure the ratios of free PSA to bound PSA show better predictive value for cancer detection. A normal value of total PSA is considered to be in the range of 0 to 4 nanograms per milliliter (ng/ml). A PSA level of 4 to 10 ng/ml is considered slightly elevated while levels between 10 and 20 ng/ml are considered moderately elevated. Concentrations above 20 ng/ml are considered highly elevated. While these values are derived from clinical evaluations, it has been shown that PSA values generally increase as men age. In addition, there are other clinical reasons for an increase in serum PSA values other than cancer. These include benign prostate enlargement, inflammation, infection, age and race. While these other conditions resulting in increased PSA concentrations may not be lethal, CaP cells have been shown to produce PSA, thus its use as a diagnostic tool.

Though prostate cancer survival rates are quite high, it is recommended that men at risk (those with a family history and those over 65) get regular PSA screening to monitor the change in PSA over time. In addition, 60% of all newly diagnosed prostate cancer cases and almost 80% of all deaths occur in men 70 years of age and older. Prostate cancer often does not cause symptoms for many years and by the time symptoms occur, the disease may have spread beyond the prostate. While regular screening can detect the presence of increased circulating PSA, there is a high incidence (60-70%) of false positives, e.g., those individuals who have elevated PSA levels, but upon biopsy, do not have a malignant condition. There is therefore a need to increase the sensitivity of the diagnosis, e.g. accurate determination of positive CaP results and the specificity of the analysis, e.g. accurate determination of the absence (negative determination or lack of false positives) of CaP. One method to control for non-malignant PSA elevation is referred to as PSA velocity. PSA velocity is simply a measure of the change in PSA elevation over time. Thus, increasing PSA levels resulting from a growing malignancy can be distinguished from chronically elevated PSA levels. However, PSA velocity is a qualitative analysis and its value is in ameliorating false positive determinations.

Traditionally, prostate cancer is described by four stages. In stage I, cancer is found in the prostate only. It is usually found accidentally during surgery for other reasons. In stage II, the cancer is more advanced, but has not spread outside the prostate. Stage III prostate cancer is identified by the spread of cancer beyond the outer layer of the prostate to nearby tissues. The cancer may be found in the seminal vesicles. Stage IV cancer is marked by the metastasis of the cancer either to nearby tissues or to distant tissues including the lymph nodes. Metastatic cancer often spreads to the bones and once the cancer has metastasized to distant tissues the prognosis for the patient is poor.

In the context of clinical diagnosis of CaP, it would be most desirable to develop a method of accurately measuring PSA levels at very low concentrations with a high level of significance, i.e. a test with a high specificity and a high sensitivity. Thus, a small increase in actual expression over time would have a high level of significance while the actual concentration is still quite low. One method to increase the detection limits of PSA would be to test for the expression of the protein and not the protein itself. Rapid progress in molecular biology has made this possible by looking for evidence of gene expression as shown by the transcription of mRNA. While all nuclei maintain a genomic copy of all genes, these genes are not expressed until transcribed into mRNA from which proteins are translated. The use of mRNA has further advantages. First, mRNA, when transcribed, is present in many copies, the number of which is directly correlated to the level of the gene's induction. Second, the polymerase chain reaction (PCR) makes possible the amplification of the message such that a very low level of message can be accurately identified. Finally, when the reaction is properly controlled, the level of the expression can be statistically quantified (quantitative PCR) such that small increases in expression as measured by mRNA transcription are significant. Thus, a more sensitive approach to the detection of low levels of circulating PSA may be the detection of mRNA coding for the PSA protein rather than the detection of the protein itself.

RT-PCR is a method whereby mRNA is reverse transcribed resulting in a cDNA copy for every RNA genomic message. This is a powerfil tool because, first, mRNA reflects the level of expression of a gene and second, the reverse transcribed cDNA is devoid of introns found in the genomic copy. Also, mRNA is generally shorter than genomic DNA and can be more easily distinguished. The use of RT-PCR in clinical oncology was first reported in 1988 when it was used to detect the bcr/abl mRNA sequence present in the chronic myelogenous leukemia cells. The rationale for RT-PCR then and now is to identify a tumor cell's specific unique mRNA sequence in order to follow the molecular progression of the disease. The tissue specific expression of the prostate specific antigen (PSA) in normal and cancerous epithelial cells makes the PSA gene expression a potentially useful marker for the detection of occult metastases of CaP.

### SUMMARY OF THE INVENTION

The invention is directed to methods whereby patients with early stage prostate cancer, that is clinically organ confined prostate cancer, are shown to possess circulating prostatic epithelial cells. In these cells there is a significant correlation of PSA expression in peripheral blood with extracapsular disease and cancer recurrence, which has great prognostic value. The detection of circulating cancer cells in the blood and bone marrow of prostate cancer patients has the potential to improve diagnosis, staging and follow-up of the disease. Further, and unlike conventional diagnostics which may be highly variable, the diagnostic ability of the presently claimed invention is highly reproducible between different laboratories.

The methods and kits on the invention represent a a significant improvement over previous methods of CaP diagnosis. According to one embodiment, prostate epithelial cells are isolated from blood and the RNA subjected to RT-PCR. The resulting cDNA is subjected to traditional PCR amplification with primers able to distinguish between the genomic copy of the gene and the cDNA copy resulting from CaP gene expression. This method provides an assay which is more sensitive, specific and reproducible as compared to conventional methods.

Results disclosed herein show that there is a correlation between the presence of prostate epithelial cells in the bone marrow with cancer recurrence. RT-PCR-PSA assays on enriched epithelial cells isolated from peripheral blood were performed. These results indicated that there is PSA expression in cells isolated from cancer patients which provide a diagnostic tool for the early detection of prostate disease.

Clearly the power of any diagnostic method is its ability to detect a malignancy at the earliest possible time. By coupling epithelial cell enrichment with RT-PCR of the PSA message (ERT-PCR/PSA), it was surprisingly determined that detection of one PSA-expressing LNCaP cell per 10⁷ lymphocytes is possible. Thus, the ability to detect significant increases in PSA expression at lower detection limits is greatly increased by the current invention. This allows for the more accurate staging of the disease, identification of risk factors and analysis of the efficacy of radical intervention than is currently available.

Other embodiments and advantages of the invention are set forth in part in the description which follows and, in part, will be obvious from this description, or may be learned from the practice of the invention.

### DESCRIPTION OF THE FIGURES

FIG. 1 shows ERT-PCR/PSA sensitivity as determined by spiking 1 ml of normal human female blood with a known number of LNCaP cells.

FIG. 2 shows the results of the ERT-PCR/PSA assay from peripheral blood of prostate cancer patients in representative experiments.

FIG. 3 shows the relationship of ERT-PCR/PSA and biopsy results in patients undergoing biopsy for suspicion of prostate cancer determination of sensitivity and specificity of analysis

### DESCRIPTION OF THE INVENTION

As embodied and broadly described herein, the present invention provides sensitive methods for the detection of prostate cancer. The invention also provides for kits and compositions used in these detection methods.

Conventional RT-PCR based assays to detect circulating prostatic epithelial cells face a technological challenge with respect to inter-laboratory as well as intra-laboratory variations resulting from the sensitivity limits of these assays. The present invention is directed to the surprising discovery that RT-PCR-PSA assays of epithelial cells enriched from peripheral blood can provide sensitive and specific early detection of prostate cancer in at-risk individuals. Further, detection of circulating prostate cancer cells in the blood and bone marrow of CaP patients have the potential to improve the staging and follow-up of the disease.

### Specific Determination of Epithelial Cell PSA Expression

Prior assays for PSA have analyzed blood for the soluble form of the PSA protein. The present invention greatly improves the sensitivity of PSA detection by analyzing blood for the presence of the mRNA precursor cell from which PSA is translated. While a preferred embodiment of the instant invention uses peripheral blood, other sources of the blood samples are equally determinative. Generally, sensitivity and specificity of the method of the invention are not dependent on the origin of the sample. The invention is not limited to blood samples, but may include samples of bodily fluids and/or tissues that contain or potentially contain epithelial cell such as bone marrow.

Since previous methods for the analysis of soluble PSA may be inconclusive or result in false positives, the present invention solves this problem by first enriching the blood sample for prostate epithelial cells. This enrichment process is most easily achieved by immuno selection although it may be achieved by other methods such as cell sorting and histologic analysis. Immuno selection of epithelial cells may comprise the selection of surface markers through the use of specific antibodies which are commercially available. Such antibodies include the Ber-EP antibody which is specific to surface epithelial glycoprotein and antibodies specific to the epithelial cell membrane antigen.

In a preferred embodiment of the present invention, antibodies specific to epithelial cell markers are coated on magnetic beads which are then added to the blood sample (Sambrook et al. Molecular Cloning: A Laboratory Manual (Third Edition) Cold Spring Harbor Press, 2001). After incubation for a period of time, preferably from two minutes to two hours, the beads are washed and adhered epithelial cells lysed. The nucleic acid is then isolated using conventional methods. Commercial reagents are also available for isolating RNA.

Briefly, magnetic beads coated with antibodies specific to epithelial cell surface antigens can be commercially obtained. Dynal Corporation (Prod. No. 161.01). After the beads are appropriately prepared, they are incubated with the blood sample for approximately 30 minutes. The cell-adhered beads are washed, the cells lysed and the lysed cells centrifuged to remove the nuclear pellet. The supernatant is then recovered and the nucleic acid extracted using phenol/chloroform extraction followed by ethanol precipitation. This provides total RNA.

mRNA can be isolated from total RNA by exploiting the polyA tail of mRNA by use of several commercially available kits. QIAGEN mRNA Midi kit (Cat. No. 70042); Promega PolyATract® mRNA Isolation Systems (Cat. No. Z5200). The QIAGEN kit provides a spin column using Oligotex Resin designed for the isolation of polyA mRNA from total RNA within 30 minutes. The Promega system uses a biotinylated oligo dT probe to hybridize to the mRNA polyA tail and requires about 45 minutes to isolate pure mRNA.

RT-PCR can be carried out using commercially available methods (e.g. Applied BioSystems (Foster City, Calif.) and Stratagene (La Jolla, Calif.; Kochanowski, Quantitative PCR Protocols: Humana Press, 1999). Total RNA may be reversed transcribed using, preferably, random hexamers and the TaqMan Reverse Transcription Reagents Kit (Perkin Elmer) following the manufactures' protocols. The cDNA is amplified using primers specific for the gene of interest. In other embodiments, RNA can be reversed transcribed using oligo(dT), which bind to the endogenous polyA tail of the mRNA. This method can be used as a universal priming means for cDNA synthesis. In yet another embodiment, the reverse transcription can be effected using primers specific to the gene sequence of interest. Thus, cDNA can be produced in a manner that is most efficacious for the examination.

After the cDNA has been synthesized from the RNA traditional PCR can be used to amplify specific sequences within the cDNA. By increasing the fidelity of the PCR reaction the sensitivity of the assay is increased such that misprimed sequences, pseudogenes and genomic contaminants are not confounding factors for the identification of occult CaP metastases. With this realization in mind, a modified form of Taq polymerase was chosen which is heat activated such that primer polymerization does not occur until the PCR reaction is at optimum temperatures. Such polymerases are commercially available from, for example, Applied BioSystems (AmpliTaq Gold, Prod. No. 4311806). Use of cDNA and the and the identification of PSA gene has afforded the use of an extremely favorable strategy to amplify desired sequences while minimizing mispriming and false positive results and the amplification of pseudogenes. Portions of the human glandular kallikrein (HGK) gene family have a high sequence homology with regions of the PSA gene. Therefore, primers were designed for regions of the PSA gene which lack homology with the HGK family. In addition, the primers are designed such that they amplify the exon ⅔ yielding a 216-bp product from the PSA mRNA and a 1.8 kb product from the genomic DNA. A nested-sense primer was then used to with the previous antisense primer and the 216 bp product to yield a 156 bp product, further confirming the accuracy of the diagnosis. Finally, to rule out amplification from genomic DNA contamination including that of pseudogenes, RNA samples were also analyzed by PCR without any RT step. PCR without any RT step did not yield any detectable product.

For the PCR reactions the sequences of the primers used are as follows:

First PCR (random hexamer primers)

Second PCR (heminested)

| | |
|---|---|
| Sense primer: | |
| 5′ -CACTGCATCAGGAACAAAAGCGT- 3′ | (SEQ ID NO 3) |
| Antisense primer: | |
| 5′ -CATCACCTGGCCTGAGGAATC- 3′ | (SEQ ID NO 4) |

As a further control, the RT-PCR reactions can be run in duplicate while a third reaction comprising a separate three μ1 RNA aliquot without reverse translation can be run with the disclosed primers to rule out contamination with genomic DNA and pseudogene interference.

As noted, these primers are designed to ensure that there is no artificial result from the presence of genomic DNA in RNA preparations as previously described by Gao et al. (Urology 53 (4), 714-721, 1999 and hereby incorporated by reference in its entirety). This method also controls for spurious priming and misamplification of genomic DNA thus limiting false positives which are a major drawback of current PSA tests. Sensitivity of the assay can be defined as the ability to detect PSA expressing cells in background lymphocytes. In the method of the invention, sensitivity was at least as low as one PSA-expressing CaP cell per 10⁷ lymphocytes which are typically found in between about 1-5 ml of blood. Preferably, sensitivity is one CaP cell in between about 10⁶ and 10⁸ lymphocytes, and more preferably, one CaP cell in between about 10⁷ and 10⁹ lymphocytes.

As an alternate definition, sensitivity of the present invention makes it possible to identify circulating prostate cancer cells in the peripheral blood of at least about 70% of prostate cancer patients, preferably at least about 80%, and more preferably at least about 90% (i.e. number of patients detected as positive by the method of the invention/number of patients with prostate cancer). Specificity, as determined by the number of true positives (i.e. true positives/true positives plus false positives), is at least about 65-70%, preferably at least about 75-80%, more preferably at least about 85-90%, and still more preferably at least about 95%.

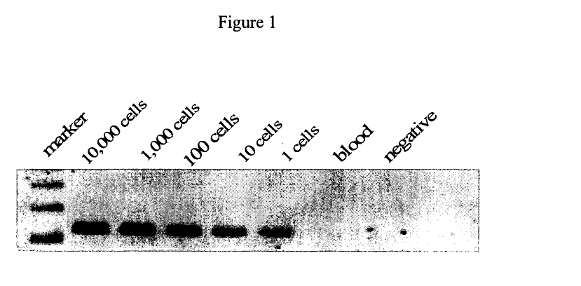

In addition, the reproducibility of ERT-PCR/PSA has been significantly improved compared to conventional methods. This method may further comprise automation and quantitation such as, for example, by using ABI 7700 machine, Taqman chemistry and a single PCR step. As illustrated in FIG. 1, ERT-PCR/PSA sensitivity was determined by spiking one ml of normal-human female blood with known numbers of LNCaP cells. The detection limit of E-PSA assay was as low as one cell in one ml of blood.

In addition, the current assay is designed such that the sensitivity of individual assays is maintained. The sensitivity is not limited to individual experiments because positive or negative scoring of each patient is based on controlled assays with two independent RT reactions and one reaction without RT for each specimen. In addition, the stringency of the sensitivity criteria of the RT-PCR assay is tested with each experiment which ensures that each batch of reagents provides the sensitivity of the detection of one CaP cell/ml of blood. This sensitivity is maintained by running a spiked control sample with each assay. Further, the detection limit is increased by performing gel-electrophoresis of the PCR product on 10% polyacrylamide gels with SYBR Gold® (Molecular Probes, Eugene, OR Cat. No. S-11494). SYBR Gold® is a modified gel stain that exhibits greater than 1,000-fold fluorescence enhancement over ethidium bromide staining. Finally, by using quantitative ERT-PCR/PSA with the Taq-man procedure, more and better information is gained on the quantity of tumor cells in blood and bone marrow. This allows a quantitative analysis of numbers of positive cells that aids in the staging of disease.

A further benefit of the invention is the ability to record a molecular profile of circulating prostate cells which may have an impact on defining prostate cancer progression and malignancy as a whole. For example, the disclosed method provides an opportunity for the molecular characterization of circulating prostate cancer cells for genomic alterations by in situ-based assays and analysis of specific genes such as p53 and bcl-2. In addition to characterization of these known cancer associated determinants, the instant invention also provides further ability to identify gene signatures of occult metastasis because once epithelial cells are isolated there is a possibility for further characterization of the circulating prostate cancer cells. Other cancer determinants may also include PSA, PSGR, DD3, PCGEM1 and PSMA genes, as well as variants of these genes that have been identified (e.g. Mikolajczyk et al., J. Urol. 161:208, 1999; Mikolajczyk et al., Urol. 50:710-714, 1997). In addition, the molecular profiling aspect of the invention with respect to one or a plurality of such genes may provide the identification of further genes whose expression changes, either up-regulated or down-regulated, during the course of the disease. Those changes may occur over a period of hours, days, months or even years. While expression of these gene may be diagnostic of cancer their changes in expression may provide extremely valuable information about the cooperative effects of gene during malignancy and/or the progress, or lack thereof, of a particular treatment. Information on the changes of expression of genes identified in this manner provide more specific data on the prognosis and ways to stage disease in future patients.

Another embodiment of the invention is directed to kits for the sensitive and specific detection of early-stage prostate cancer such as, for example, a subclinical stage of prostate cancer or organ-confined prostate cancer. Kits comprise reagents for conducting an RT-PCR on nucleic acid isolated from a sample of blood. Reagents include a Taq polymerase enzyme and additional enzyme capable of detectably amplifying a single gene in a single cell in a background of 10⁷ cells. Reagents further include a set of primers such as, for example, random-hexamer primers, preferably sequences specific for exons of the PSA gene, more preferably oligo-dT primers, and still more preferably SEQ ID NO 1 and SEQ ID NO 2, and a set of PSA-specific primers, preferably a hemi-nested set of primers, and more preferably SEQ ID NO 3 and SEQ ID NO 4. Reagents may further include additional solutions, buffers, and materials necessary or appropriate in conventional ERT-PCR/PCR, and typical prostate cancer detection kits. Kits of the invention are capable of detecting one PSA-expressing cell in one ml of blood or 10⁷ lymphocytes. Sensitivity of kits is preferably at least about 80% or greater, and specificity is preferably at least about 85% or greater.

The following examples are offered to illustrate embodiment of the present invention, but should not be viewed as limiting the scope of the invention.

### EXAMPLES

### Example 1

### Immunomagnetic Capture of Epithelilal Cells from Blood and RNA Preparation

Five milliliters of blood was collected into a sodium citrate tube and transported to the laboratory on wet ice within 2-3 hours after the blood was drawn. Dynabeads coated with monoclonal antibody, Ber-EP4 specific for immunomagnetic enrichment of human epithelial cells (Dynal, product number: 116.02), were mixed thoroughly in the vial. Eighty micro-liters of Dynabeads suspension was transferred to a 0.5 ml Eppendorf tube, placed on a Dynal MPC rack for 2 minutes and the supernatant pipetted off. One-half ml of cold washing buffer [2% heat inactivated fetal bovine serum (FBS) in phosphate-buffered saline (PBS)] was added to the beads, the tube was removed from the rack, beads were resuspended and the supernatant was removed by placing the suspension in the MPC rack as before. Beads were finally suspended in 80 μl of the washing buffer and added into 5 ml of blood followed by gentle tilting and rotation for 30 minutes at 4° C. on tube rotator allowing the capture of epithelial cells by beads via the Ber-EP4 antibody. By placing the tube containing blood and beads in the Dynal MPC rack for 15 minutes the beads were separated by carefully pipetting off the blood. Dynabeads were washed twice with 5 ml of cold washing buffer and once in 0.5 ml of cold buffer and transferred to an Eppendorf tube. The supernatant was pipetted off by placing the tube in Dynal MPC rack. The rosetted cells on the beads were lysed by 0.3 ml RNAzol B (Tel-TEST, Inc). RNA was prepared according to the manufacture's recommendations and 2 μl glycogen was added as carrier prior to isopropyl alcohol precipitation. RNA pellet was dissolved in 10 μl DEPC water and stored at −80° C. Three til of RNA was used for each RT-PCR reaction, performed in duplicate for each patient with a third RNA sample used as a control (e.g. Example 5).

Reverse Transcriptase (RT)-Polymerase Chain Reaction (PCR) for PSA Gene Expression:

Three μl of total RNA was reverse transcribed into each cDNA using random hexamer primer and MuLV reverse transcriptase (Perkin Elmer lot B06283) in a final 20 μl volume according to the supplier's recommendation. Five microliters of this cDNA served as starting template for the first PCR reaction. The 25 μl volume of the PCR reaction mixture included 1.0 mM MgCl₂, 200 μM deoxy-nucleotide triphosphate (dNTP), 25 ng of each primer, and 0.5 U AmpliTaq Gold Polymerase (Perkin Elmer Lot B00783). PCR amplification protocol included one cycle at 95° C. for 10 minutes, 25 cycle at 94° C. for 30 seconds, 66° C. for 1 minute and 72° C. for 1 minutes, followed by one cycle at 72° C. for 5 minutes. Two microliters of the first PCR reaction served as the template for the hemi-nested PCR using one internal primer and one original primer related to the first set of PCR primers in 50 μl reaction volume. The PCR components and cycles for nested PCR were the same as for first PCR reaction.

PCR Primers:

PCR primers were designed using PrimerDetect software (Clonetech,CA) and their specificity were defined by sequence homology searches of NCBI sequence database to exclude the possibility of amplification of PSA related genes. This is an important consideration as PSA belongs to the family of kallikrein gene family. For the first PCR reaction, the sense primer (5′5′ GCCTCTCGTGGCAGGGCAGTC-3′; SEQ ID NO 1) and the antisense primer (5′-CATCACCTGGCCTGAGGAATC-3′; SEQ ID NO 2) were selected from exon ⅔, which yield a 216-bp product from PSA RNA and a 1.8 kb product from the PSA genomic DNA. Therefore, we could easily determine the contamination of genomic DNA in RNA preparation. The Nested PCR sense primer (5′ CACTGCATCAGGAACAAAAGCGT-3′; SEQ ID NO 3) and the antisense primer (5′-CATCACCTGGCCTGAGGAATC-3′; SEQ ID NO 4) yield a 156-bp DNA fragment.

Three μl RNA aliquots from each patient sample were processed for two identical RT-PCR reactions and one control reaction with out RT (NO-RT) to rule out a false positive result with genomic DNA and pseudogene interference. PCR controls also included a no template-PCR reaction as a negative control and a positive control LNCaP RNA to validate the assay. The analysis of the RT-PCR-derived PSA fragment included (a) visual detection of the expected size DNA bands by SYBR GOLD staining of the TBE-10% Polyacrylamide gel from hemi-nested PCR reactions; (b) direct DNA sequencing of randomly selected PCR products confirming their identity as PSA and (c) inclusion of a positive control in each assay to define the sensitivity of the assay as one PSA-expressing LNCaP cells/ml blood.

Specificity of the Ampli Taq Gold DNA polymerase and reverse transcriptase enzymes was considered crucial for this experiment as we noticed that different batches of enzymes showed varying sensitivity. It was important to select the enzyme lot that reproducibly detected one PSA expressing LNCaP cell per ml of female blood.

### Example 2

### Confirmation of Assay Sensitivity

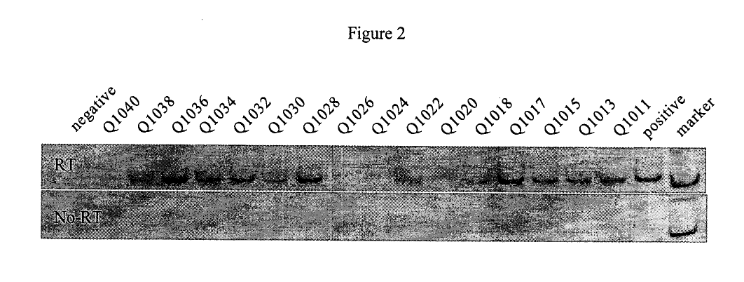

To confirm the sensitivity of the protocol, 1 ml of normal human female blood was spiked with a known number of LNCaP cells. The detection limit of ERT-PCR/PSA assay was as low as one cell in 1 ml of blood. Briefly, sensitivity of the ERT-PCR/PSA assay was determined by serial dilutions of total RNA derived from a known number of LNCaP cells spiked into total RNA of a known number of peripheral blood mononuclear cells (PBMC) at LNCaP/PBMC cell ratios of 1:10², 1:10³, 1:10⁴, 1:10⁵, 1:10⁶, 1:10⁷, 1:10⁸, 1:10⁹. The PCR amplification product was detected by ethidium bromide staining of agarose gels. The limit of ERT-PCR/PSA in this assay was 1:10⁷. The sensitivity of the assay from the analysis of peripheral blood was illustrated in representative experiments which are shown in FIG. 2.

Using primers specific for the PSA gene, sensitivity was defined as the detection of the number of prostate cancer cells (one LNCaP cell) per ten million lymphocytes (RNAzol B; RNA extraction kit; Tel-Test, Inc.). Control samples consisted of 20 healthy volunteers (2 women and 18 men). Peripheral blood samples were negative by RT-PCR in each control. Nested PCR product was analyzed on an ethidium bromide stained agarose gel. To confirm the identity of RT-PCR product as PSA, randomly selected PCR products were sequenced.

### Example 3

### Analysis of PSA Expressing Cells in Peripheral Blood of Prostate Cancer Patients

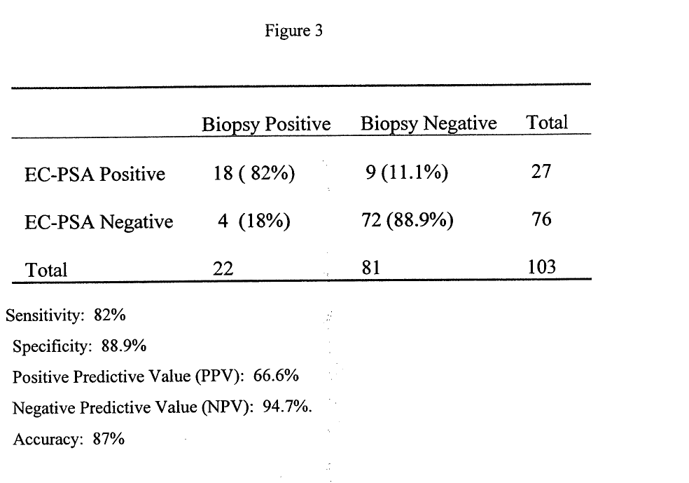

In 85 patients undergoing radical prostatectomy, a minimum of two independent ERT-PCR/PSA assays detected circulating prostate cells preoperatively in 27 patients (31.8%). In 12 locally advanced or advanced stage patients, ERT-PCR/PSA was positive in 5 patients (41.7%), and in 22 controls, no patient was ERT-PCR/PSA positive. In 10 randomly selected cases, the RT-PCR product was confirmed as PSA by DNA sequencing. Of the 27 RT-PCR positive RP patients, 11 of 27 (40.7%) had non-organ confined disease (≧pT3a), and of the 58 RT-PCR negative patients, 32 (55.2%) had non-organ confined disease. RT-PCR positive patients also had lower margin positivity (9 of 27, 33.3%) than did RT-PCR negative patients (21 of 58, 36.2%). Finally, at a mean follow-up of 25.7 months, 5 of 27 (18.5%) RT-PCR positive patients recurred (PSA) compared to 14 of 58 (24.1%) RT-PCR negative patients. The results of representative experiments showing positive identification of CaP from the peripheral blood of patients undergoing biopsy for suspicion of prostate cancer is shown in FIG. 3.

On the basis of this blinded study, RT-PCR for PSA expressing cells in 85 pre-radical prostatectomy patients is not related to clinical stage, age, race, grade, Gleason sum, serum PSA or PAP, tumor volume, or tumor multifocality. RT-PCR positivity did not predict pathologic stage or early PSA recurrence.

### Example 4

### Analysis of PSA Expressing Cells in Bone Marrow of Prostate Cancer Patients

RNA from bone marrow aspirates of patients undergoing radical prostatectomy were analyzed for PSA gene expression. Fifty-one of 116 patients (44.0%) were positive resulting in a minimum of two independent RT-PCR assays, whereas 77/116 (66%) of the patients were RT-PCR positive in at least one assay. All of these RNA specimens, whether scoring positive or negative in the RT-PCR assay, show comparable levels of glyceraldehyde phosphate dehydrogenase (GAPDH) gene expression by RT-PCR/GAPDH assay. On the basis of this blind study, ERT-PCR/PSA positivity did not correlate with age, race, acid phosphatase, grade, Gleason sum, or clinical stage categories. In 116 radical prostatectomy patients categorized by pathologic stage, 26 of 51 (51.0%) of patients with non-organ confined disease (stage pT3a or greater) were positive in BM-RT-PCR/PSA as compared to 33 of 65 (49.0%) patients with organ confined disease. In an examination of early disease-free survival rates by RT-PCR assay, 8 (15.7%) experienced a PSA recurrence compared to 2 of 65 (3.1%) of patients with a negative assay. Kaplan-Meier analysis determined that the 2-year disease-free survival was 96.6 versus 77.5% for RT-PCR negative and positive patients, respectively (p=0.054). Furthermore, in this cohort, bone marrow RT-PCR/PSA was superior to the variables of PSA and radical prostatectomy Gleason sum. In multivariate Cox regression analysis with the four prognostic factors of pre-treatment PSA, Gleason sum, pathologic stage and BM-RT-PCR, RT-PCR and pathological stage status remained significant (p<0.05) while PSA and Gleason did not remain significant.

### Example 5

### Value of RT-PCR of PSA-Expressing Cells in Patients with Prostate Cancer

In an effort to clarify the value of RT-PCR of PSA-expressing cells in patients with prostate cancer, a comprehensive and blinded evaluation of the RT-PCR PSA positivity in blood and bone marrow of patients undergoing radical prostatectomy was performed. Circulating epithelial cells were isolated from the peripheral blood using anti-epithelial cell antibody Ber-EP4 coated magnetic beads (Dynal Corporation, Prod. No. 161.02). Total RNA prepared from enriched epithelial cells fractionated on the beads was analyzed for the expression of glyceraldehyde-3-phosphate dehydrogenase (GAPDH) and PSA using nested RT-PCR assays. Peripheral blood specimens of 111 of 137 (81.6%) (pre-surgery) prostate cancer patients who underwent radical prostatectomy were positive in ERT-PCR/PSA assay. Reproducibility of ERT-PCR/PSA assay was 83.8% (93 of 111) in specimens of cancer patients in two independent RT-PCR assays. Peripheral blood specimens from 13 of 79 patients who had biopsy for suspicion of prostate cancer were positive in ERT-PCR/PSA assay. Thirteen of 15 patients (87%) with biopsy proven prostate cancer and 58 of 64 patients with biopsy negative for prostate cancer (90%) were negative in ERT-PCR/PSA assay. These results are illustrated in FIG. 3. These results show that a high fraction of patients with clinically organ confined prostate cancer contain circulating prostatic epithelial cells. To further control for false positives a third aliquot of RNA isolated from each subject was subjected to PCR without a reverse transcriptase reaction. The purpose of this step was to control from the presence of the genomic PSA copy or pseudogenes which may give a product from the primers previously described. These results also suggest that ERT-PCR/PSA assay along with the similar assays for other prostate epithelial cell specific genes may be a potentially useful tools in the detection of prostate cancer. These observations underscore new diagnostic application of circulating prostate cells (see Urology 53, 714-721, 1999 and Journal of Urology 161, 1070-1076, 1999; both of which are hereby specifically and entirely incorporated herein by reference).

Other embodiments and uses of the invention will be apparent to those skilled in the art from consideration of the specification and practice of the invention disclosed herein. All references and written information cited herein for whatever reason, including all priority documents, are entirely and specifically incorporated by reference. It is intended that the specification and examples be considered exemplary only.

1. A sensitive and specific method for detection of prostate cancer in a patient comprising:
isolating epithelial cells from a sample of blood obtained from said patient;
lysing said epithelial cells and isolating nucleic acid;
conducting RT-PCR on said isolated nucleic acid wherein said RT-PCR comprises:
synthesizing cDNA from said nucleic acid with a set of primers and a Taq polymerase enzyme capable of detectably amplifying a single gene in a single cell in a background of 10⁷ cells; and
conducting PCR on said cDNA wherein said PCR comprises:
synthesizing DNA from said cDNA with a set of PSA-specific primers and additional Taq polymerase enzyme capable of detectably amplifying a single gene in a single cell in a background of 10⁷ cells;
wherein said method is capable of detecting one PSA-expressing cell in one ml of blood or 10⁷ lymphocytes.

2. The method of claim 1 wherein sensitivity is at least about 80% or greater.

3. The method of claim 1 wherein specificity is at least about 85% or greater.

4. The method of claim 1 wherein the prostate cancer detected is at a subclinical stage.

5. The method of claim 4 wherein the subclinical stage is organ-confined prostate cancer.

6. The method of claim 1 wherein the patient is a human.

7. The method of claim 1 wherein the epithelial cells are isolated by an immnunological technique.

8. The method of claim 7 wherein the immunological technique is selected from the group consisting of immunoprecipitation, immuno-affinity chromatography, dynabead enrichment and combinations thereof.

9. The method of claim 7 wherein the immunological technique comprises antibodies specific to epithelial antigens.

10. The method of claim 9 wherein the epithelial antigens are selected from the group consisting of Ber-EP4 antigens, epithelial membrane antigens, epithelial cell-surface antigens, and combinations thereof.

11. The method of claim 1 wherein the sample of blood comprises peripheral blood.

12. The method of claim 1 wherein the sample of blood comprises a volume of 5 ml or less.

13. The method of claim 1 wherein the set of primers comprises random-hexamer primers.

14. The method of claim 1 wherein the set of primers contains sequences specific for exons of the PSA gene.

15. The method of claim 1 wherein the set of primers comprising oligo-dT primers for the amplification of total mRNA.

16. The method of claim 1 wherein the set of PSA-specific primers comprise a hemi-nested set of primers.

17. The method of claim 1 wherein the set of PSA-specific primers comprise SEQ ID NO 3 and SEQ ID NO 4.

18. The method of claim 1 wherein the set of primers comprise sequences from a gene that has an elevated expression in CaP cells as compared to non-cancerous cells.

19. The method of claim 18 wherein the gene is selected from the group consisting of PSGR, DD3, PCGEM1, p53, bcl-2, hK², PSMA, and combinations thereof.

20. A sensitive and specific method for detection of prostate cancer in a patient comprising:
isolating epithelial cells from a sample of blood obtained from said patient;
lysing said epithelial cells and isolating nucleic acid;
conducting RT-PCR on said isolated nucleic acid wherein said RT-PCR comprises:
synthesizing cDNA from said nucleic acid with a set of primers and a Taq polymerase enzyme capable of detectably amplifying a single gene in a single cell in a background of 10⁷ cells; and
conducting PCR on said cDNA wherein said PCR comprises:
synthesizing DNA from said cDNA with a set of PSA-specific primers and additional Taq polymerase enzyme capable of detectably amplifying a single gene in a single cell in a background of 10⁷ cells;
wherein said method is capable of detecting one PSA-expressing cell in one ml of blood or 10⁷ lymphocytes and sensitivity is greater than 80% and specificity is greater than 85%.

21. A kit for the sensitive and specific detection of early-stage prostate cancer comprising:
reagents for conducting RT-PCR on nucleic acid isolated from a sample of blood wherein said reagents comprises a Taq polymerase enzyme capable of detectably amplifying a single gene in a single cell in a background of 10⁷ cells and a set of primers;
reagents for conducting PCR on cDNA wherein said reagents comprises additional Taq polymerase enzyme capable of detectably amplifying a single gene in a single cell in a background of 10⁷ cells and a set of PSA-specific primers,
wherein said kit is capable of detecting one PSA-expressing cell in one ml of blood or 10⁷ lymphocytes.

22. The kit of claim 21 wherein sensitivity of the method is at least about 80% or greater.

23. The kit of claim 21 wherein specificity of the method is at least about 85% or greater.

24. The kit of claim 21 wherein the early-stage prostate cancer detected is a subclinical stage.

25. The kit of claim 24 wherein the subclinical stage is organ-confined prostate cancer.

26. The kit of claim 21 wherein the patient is a human.

27. The kit of claim 21 wherein the set of primers comprises random-hexamer primers.

28. The kit of claim 21 wherein the set of primers contains sequences specific for exons of the PSA gene.

29. The kit of claim 21 wherein the set of primers comprising oligo-dT primers for the amplification of total mRNA.

30. The kit of claim 21 wherein the set of primers comprises SEQ ID NO 1 and SEQ ID NO 2.

31. The kit of claim 21 wherein the set of PSA-specific primers comprise a hemi-nested set of primers.

32. The kit of claim 21 wherein the set of PSA-specific primers comprise SEQ ID NO 3 and SEQ ID NO 4.

33. A kit for the sensitive and specific detection of early-stage prostate cancer comprising:
reagents for conducting RT-PCR on nucleic acid isolated from a sample of blood wherein said reagents comprises a Taq polymerase enzyme capable of detectably amplifying a single gene in a single cell in a background of 10⁷ cells and a set of primers; and
reagents for conducting PCR on said cDNA wherein said reagents comprises additional Taq polymerase enzyme capable of detectably amplifying a single gene in a single cell in a background of 10⁷ cells and a set of PSA-specific primers,
wherein sensitivity of said method is greater than 80% and specificity is greater than 85%.

34. A method for determining a molecular profile of a prostate cell comprising:
isolating epithelial cells from a sample containing said prostate cell;
lysing said epithelial cells and isolating nucleic acid;
conducting RT-PCR on said isolated nucleic acid wherein said RT-PCR comprises:
synthesizing cDNA from said nucleic acid with a set of primers and a Taq polymerase enzyme capable of detectably amplifying a single gene in a single cell in a background of 10⁷ cells; and
conducting PCR on said cDNA wherein said PCR comprises:
synthesizing DNA from said cDNA with a set of primers specific for a cancer-determinant gene and additional Taq polymerase enzyme capable of detectably amplifying a single gene in a single cell in a background of 10⁷ cells; and
determining the molecular profile of the cancer-determinant gene for said prostate cell.

35. The method of claim 34 wherein the cancer-determinant gene is selected from the group consisting of genes which encode PSA, p53, bcl-2, PSGR, DD3, PCGEM1, PSMA and variants thereof.

36. The method of claim 34 which determines the molecular profile of a plurality of cancer-determinant genes.

37. The method of claim 34 which is performed multiple times with multiple samples all obtained from the same source, but at different times over a set time period, to determine the progression of said molecular profile over said set time period.
